(19) United States
(12) Patent Application Publication
(10) Pub. No.: US 2024/0245058 A1
(43) Pub. Date: Jul. 25, 2024

(54) METHOD FOR BIOCONTROL OF FUSARIUM HEAD BLIGHT IN CEREALS

(71) Applicants: UNIVERSITE PARIS-SACLAY, Saint Aubin (FR); CENTRE NATIONAL DE LA RECHERCHE SCIENTIFIQUE - CNRS -, PARIS (FR); B 2 B, PARIS (FR)

(72) Inventors: Marie DUFRESNE, ORSAY (FR); Julie OBERT, ORSAY (FR); Tracy PLAINCHAMP, ORSAY (FR); Olivier BESNARD, PARIS (FR); Jean-Marc SENG, ORSAY (FR)

(73) Assignees: UNIVERSITE PARIS-SACLAY, Saint Aubin (FR); CENTRE NATIONAL DE LA RECHERCHE SCIENTIFIQUE - CNRS -, PARIS (FR); B 2 B, PARIS (FR)

(21) Appl. No.: 17/260,729

(22) PCT Filed: Jun. 19, 2019
(86) PCT No.: PCT/EP2019/069596

§ 371 Date: Jan. 15, 2021

(30) Foreign Application Priority Data
Jul. 19, 2018 (FR) 1856722

(51) Int. Cl. A01N 63/38; A01P 3/00; C12N 1/14; C12R 1/885

(52) U.S. Cl. CPC A01N 63/38; A01P 3/00; C12N 1/14; C12R 2001/885

(57) ABSTRACT

A novel Trichoderma harzanium strain, filed on Jun. 12, 2018 with the CNCM (Collection Nationale de Cultures de Micro-organismes), 25-28 rue du Docteur Roux, 75724 Paris Cedex 15, under number CNCM I-5327. Also, the use of this Trichoderma harzanium strain to control fusarium head blight in cereals selected from wheat, Brachypodium distachyon, triticale, oats and rye, and more particularly to combat fusarium head blight in wheat.

The present invention relates to a method for biocontrol of Fusarium head blight in cereals, and in particular Fusarium wheat head blight, by using a strain of the antagonistic fungus Trichoderma sp.

Biocontrol is defined as a method of crop protection based on the use of macroorganisms, microorganisms, natural substances and chemical mediators (such as pheromones).

The desire to reduce agriculture's dependence on synthetic pesticides is increasingly stimulating innovation in the field of biocontrol.

Fusarium infections are common fungal diseases in cereals, caused in particular by certain fungi of the genera Fusarium and Microdochium. The development of these diseases in cereal crops varies considerably depending on the species and varieties considered and depending on the climatic conditions at the time of heading and the beginning of flowering.

Fusarium wheat head blight may cause large losses of yield (between 20 and 40% of the yield in intensive agriculture), but it may in particular cause contamination of the grains by the production of mycotoxins (such as the mycotoxin “deoxynivalenol” or “DON”) and therefore degradation of the food quality of wheat grain.

Studies into the use of Trichoderma for protecting wheat crops against Fusarium head blight (FHB), caused by a complex of species such as Fusarium graminearum and Microdochium nivale (M. nivale nivale and M. nivale majus), have already been described in the literature.

Thus, preventive application on the wheat head with Trichoderma harzianum was carried out by the Indian team of Panwar et al. (2014)¹. The authors observed a certain level of protection against Fusarium head blight and proposed a combination of two species of Trichoderma.

However, it is difficult to combine two species on an industrial scale, in view of the difficulties of standardizing the production of several live microorganisms within one and the same preparation. Furthermore, the Trichoderma strains are Indian and the current recommendations with respect to European regulations are to use strains obtained from the country of origin (indigenous strains), in this case strains originating from European countries. It is in fact important to be able to use indigenous strains, in order to limit the proliferation of undesirable organisms.

The work by Mahmoud (2016)² describes the use of Trichoderma harzianum and Bacillus subtilis against Fusarium wheat head blight.

Once again, the strains isolated are Egyptian, which compromises use in Europe. Furthermore, the test setup described in Mahmoud (2016) is far from agricultural reality since the antagonist (T. harzianum and/or B. subtilis) is inoculated at the same time as the Fusarium pathogen (artificial contamination), which is never the case in wheat growing since the two microorganisms have their own biology and will be applied on the heads at different times.

Finally, the Italian work by Sarrocco et al. (2013)³ describes the use of Trichoderma gamsii against Fusarium wheat head blight.

However, the species T. gamsii has a major drawback: its sporulation is insufficient to envisage aerial applications in the open. Thus, only incorporation in the soil is currently envisaged in Italy for combating early attacks of Fusarium.

There is therefore still a need to develop new strains of Trichoderma sp. for combating Fusarium head blight in cereals, and in particular Fusarium wheat head blight.

One aim of the invention is to isolate one or more new strains of the antagonistic fungus Trichoderma sp., which have Europe as their geographic origin, and preferably France, and which make it possible in particular to combat Fusarium wheat head blight.

Another aim of the invention is to isolate one or more new strains of Trichoderma sp. that can be applied by aerial application (as opposed to application on the soil) on cereal crops, and in particular wheat crops.

Yet another aim of the invention is to isolate one or more new strains of Trichoderma sp. that can replace (at least partially) the fungicidal chemicals of the azole type currently on the market for combating Fusarium head blight in cereals, and in particular Fusarium wheat head blight.

Another aim of the invention is to isolate one or more new strains of Trichoderma sp. that can be used in combination with fungicides of the azole type, with the aim of significantly reducing the recommended approved dose of fungicide for effective action for combating Fusarium head blight in cereals, and in particular Fusarium wheat head blight.

In general, the invention has the aim of finding a new method for biocontrol of Fusarium head blight in cereals, and in particular Fusarium wheat head blight, which would be a method that is alternative or complementary to the methods of combating Fusarium head blight that mainly employ fungicides of the azole type.

After intensive research conducted by the inventors on the fungus Trichoderma sp, they succeeded in isolating, from soils for wheat growing in France, strains of Trichoderma, and more particularly a strain of Trichoderma harzianum that displays very effective action for combating Fusarium head blight in cereals, and in particular Fusarium wheat head blight.

This strain will also be usable, advantageously, with fungicides of the azole type.

The invention relates firstly to a new strain of Trichoderma harzianum.

The invention relates secondly to the use of this new strain of Trichoderma harzianum for combating Fusarium head blight in cereals, and in particular Fusarium wheat head blight.

The invention further relates to a method of combating Fusarium head blight in cereals, and in particular Fusarium wheat head blight, characterized in that it comprises a step of bringing the new strain of Trichoderma harzianum into contact with the cereals.

The invention further relates to a composition for combating Fusarium head blight in cereals, said composition comprising the Trichoderma harzianum strain of the invention, a formulating agent and/or an adjuvant, and optionally a fungicide of the azole type.

The invention further relates to a kit comprising, separately, the Trichoderma harzianum strain of the invention and a fungicide of the azole type.

The new strain of the invention is more particularly the Trichoderma harzianum strain deposited on Jun. 12, 2018 at the National Collection of Cultures of Microorganisms (CNCM), 25-28 rue du Docteur Roux, 75724 Paris Cedex 15, under number CNCM I-5327.

In the course of research conducted by the inventors, the latter also isolated the following strains:
Trichoderma harzianum deposited on Jun. 12, 2018 at the CNCM under number CNCM I-5326,
Trichoderma longibrachiatum deposited on Jun. 12, 2018 at the CNCM under number CNCM I-5328.

However, it was found that these strains are of much lower efficacy on Fusarium wheat head blight compared to that of the strain CNCM I-5327 (see example 2).

For brevity, the aforementioned strains will be called respectively hereinafter:
“Tr B” for the Trichoderma harzianum strain of the invention, deposited on 06/12/18 under number CNCM I-5327,
“Tr A” for the strain deposited on 06/12/18 under number CNCM I-5326, and
“Tr C” for the strain deposited on 06/12/18 under number CNCM I-5328.

The expression “strain” in the present application denotes more particularly a “strain of filamentous fungus”. “Strain of filamentous fungus” means an individual of a given species comprising identical nuclei within one and the same cytoplasm. These identical nuclei endow the strain with identical and stable morphological, physiological and ecological properties. The strain is obtained after regeneration of a spore. If this spore is uninucleate, which is the case for Trichoderma, it is then called a clone.

The present invention also relates to the use of the strain Tr B for combating Fusarium head blight of cereals selected from the group comprising wheat, Brachypodium distachyon, triticale, oat, rye and maize, and preferably wheat.

Wheat, Brachypodium distachyon, triticale, oat, rye, and maize are cereals in the Poaceae family (Poaceae).

The wheat cited above denotes both soft wheat and hard wheat. As examples of varieties of soft wheat, we may mention varieties of winter soft wheat such as Rubisko, Cellule, Apache and Boregar.

As examples of varieties of hard wheat, we may mention varieties such as Babylone, Miradoux and Sculptur.

According to the invention, “combating” means a method of preventing and/or treating Fusarium head blight in cereals, and preferably a method of preventing Fusarium head blight of wheat.

“Method of preventing” means, in the sense of the invention, complete or partial prevention. Complete prevention will make it possible to prevent the cereals being contaminated by Fusarium head blight once the Trichoderma strain of the invention has been applied on the cereals. Partial prevention will not prevent Fusarium head blight developing, but the symptoms caused by Fusarium head blight will be less severe and/or fewer heads will be contaminated with Fusarium head blight.

The strain Tr B of the invention is particularly effective for combating Fusarium head blight caused by a fungal phytopathogen of the genus Fusarium and/or Microdochium.

As examples of phytopathogenic agents of the genus Fusarium, we may mention those selected from the group comprising Fusarium graminearum, Fusarium culmorum, Fusarium poae, Fusarium sporotrichioides, Fusarium equiseti and mixtures thereof.

As examples of phytopathogenic agent of the genus Microdochium, we may mention Microdochium nivale, and more particularly the two subspecies M. nivale majus and M. nivale nivale.

According to an advantageous embodiment of the invention, the strain Tr B is particularly effective for combating Fusarium wheat head blight.

According to yet another advantageous embodiment of the invention, the strain Tr B is particularly effective for decreasing the content of mycotoxins present in wheat, said mycotoxins in particular being deoxynivalenol (DON).

In fact, certain phytopathogenic agents of the genus Fusarium, for example such as F. graminearum, are responsible for producing mycotoxins such as DON.

In the European Union the maximum values permitted for the commonest mycotoxins, such as DON, in agricultural and food products were established by Regulation 1881/2006.

Thus, in consequence, the Trichoderma strain Tr B of the invention therefore also makes it possible to combat Fusarium wheat head blight by lowering the content of DON mycotoxins present in wheat.

According to another embodiment of the invention, application of the strain Tr B will be carried out at the level of the cereal head, and preferably the wheat head.

More particularly according to the invention, application of the strain Tr B will preferably be carried out starting from the beginning of flowering of the cereals and until full flowering of the cereals.

In other words, application of the strain Tr B will advantageously be carried out starting from the BBCH 61 stage and up to the BBCH 65 stage.

Preferably according to the invention, one or two applications of the strain Tr B are sufficient for effective action against Fusarium head blight in cereals, and in particular Fusarium wheat head blight.

The “BBCH” scale (“BBCH” is the German acronym “Biologische Bundesanstalt Bundessortenamt und CHemische Industrie”) of cereals describes the phenological development of cereals using the BBCH code.

The stage “BBCH 61” corresponds to the beginning of flowering; the first anthers are visible.

The stage “BBCH 65” corresponds to full flowering, with 50% of the anthers showing.

For a cereal such as wheat, the main risk of epidemic caused by the phytopathogen Fusarium or Microdochium occurs at full flowering of the cereals, at the BBCH 65 stage.

However, epidemics are not always synchronous and may be spread over time.

According to an advantageous embodiment of the invention, the strain Trichoderma Tr B is applied from the beginning of flowering of the cereals, several days before the BBCH 65 stage.

According to yet another advantageous embodiment of the invention, the strain Tr B is applied on the cereals, at a concentration of spores ranging from 10⁶ to 10⁹ spores/ml, and preferably from 10⁷ to 10⁸ spores/ml, in a volume of 100 to 200 liters per hectare.

It is also possible according to the invention to use the Trichoderma strain Tr B in combination with a fungicide of the azole type selected from the group comprising prothioconazole, tebuconazole, metconazole, mefentrifluconazole and mixtures thereof.

The invention thus further relates to the use of the Trichoderma strain Tr B for separate or sequential administration with said fungicide of the azole type.

According to the invention, the fungicide will preferably be applied after application of the strain Tr B on the heads.

As an example, the strain Tr B of the invention may be applied firstly on the wheat head at the BBCH 61 stage, and then a fungicide of the azole type may be applied secondly on the wheat head at the BBCH 65 stage.

According to one embodiment of the invention, when the strain Tr B of the invention is used in combination with the fungicide of the azole type, the amount of fungicide is from 2% to 60%, and preferably from 4% to 35% of the recommended dose for effective action for combating Fusarium head blight.

In other words, this signifies that when the fungicide of the azole type is combined with the strain Tr B of the invention, the amount thereof recommended for effective action for combating Fusarium head blight can be reduced by 40% to 90% for an action that is at least as effective, or even more effective, than if the fungicide had been used at the recommended dose.

“Recommended dose” means the dose usually employed in agricultural practice. Thus, the commercial specialties based on tebuconazole are approved at 1 l/ha (liter per hectare), but the recommended doses are instead 0.8 l/ha, or even 0.75 l/ha.

The approved dose is the maximum dose permitted per treatment for a phytosanitary product (in this case fungicide) on a crop and for a target organism (for example a parasite or a rodent).

The combination of the strain Tr B of the invention with the fungicide of the azole type is therefore particularly advantageous as it allows an appreciable reduction in the amounts of fungicide recommended for effective action.

Finding new means for reducing the doses of pesticides by combining them with other products complies in particular with the thematic strategy of the European Union of 2006 (http://data.europa.eu/eli/dir/2009/128/oj) on the sustainable use of pesticides.

The present invention also relates to a composition for combating Fusarium head blight in cereals characterized in that it comprises:
the Trichoderma harzianum strain Tr B, and
a formulating agent and/or an adjuvant.

The formulating agent and/or adjuvant is one of those usually employed in compositions intended for agricultural use.

The composition described above may be defined as a crop protection composition based on the use of live organisms and thus responds to the desire to reduce agriculture's dependence on synthetic pesticides.

The invention also relates to a kit, characterized in that it comprises, separately:
the Trichoderma harzianum strain Tr B,
a fungicide of the azole type selected from the group comprising prothioconazole, tebuconazole, metconazole, mefentrifluconazole and mixtures thereof.

The present invention also relates to a method of combating Fusarium head blight in cereals, and preferably Fusarium wheat head blight, said method being characterized in that it comprises a step of contacting the Trichoderma harzianum strain Tr B with the cereals.

As stated above, said contact between the strain Tr B and the cereals takes place at the level of the cereal head, and preferably the wheat head.

According to an advantageous embodiment of the invention, in the method of combating Fusarium head blight in cereals, the step of contacting the strain Tr B with the cereals is carried out starting from the beginning of flowering of the cereals and until full flowering of the cereals, or starting from the BBCH 61 stage and up to the BBCH 65 stage.

The method of combating Fusarium head blight in cereals, and preferably Fusarium wheat head blight, is also characterized in that it further comprises a step of contacting the cereals with a fungicide of the azole type as defined above, said fungicide being applied separately or sequentially with the strain Tr B.

According to said method, the fungicide will preferably be applied after application of Trichoderma on the heads.

The invention also relates to a method of combating Fusarium head blight in cereals, and preferably Fusarium wheat head blight, said method being characterized in that it comprises a step of bringing the cereals into contact with a composition comprising:
the strain Tr B, and
a formulating agent and/or an adjuvant.

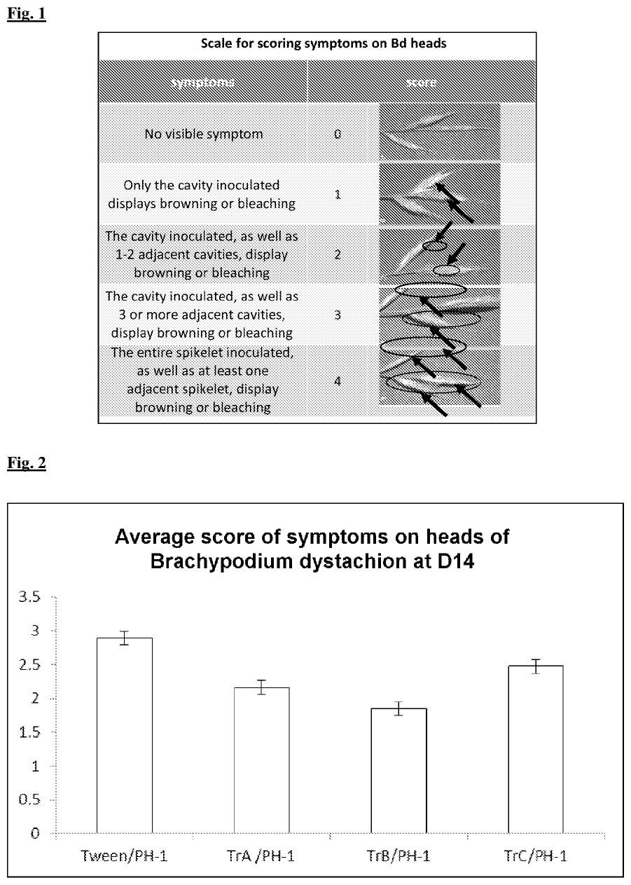

The invention will be better understood in light of the following examples, which are nonlimiting and purely for purposes of illustration. FIGS. 1 to 6 illustrate the examples, given hereunder.

FIG. 1 is a scale for scoring symptoms (scores 0, 1, 2, 3 or 4) on heads of Brachypodium distachyon (wild ecotype Bd21-3) inoculated with the strain PH-1 of Fusarium graminearum.

FIG. 2 is a comparison of the scores observed 14 days after inoculation of the strain PH-1 of Fusarium graminearum on Brachypodium distachyon (wild ecotype Bd21-3), untreated, or treated with the strain of the invention Tr B and the comparative Trichoderma strains Tr A and Tr C.

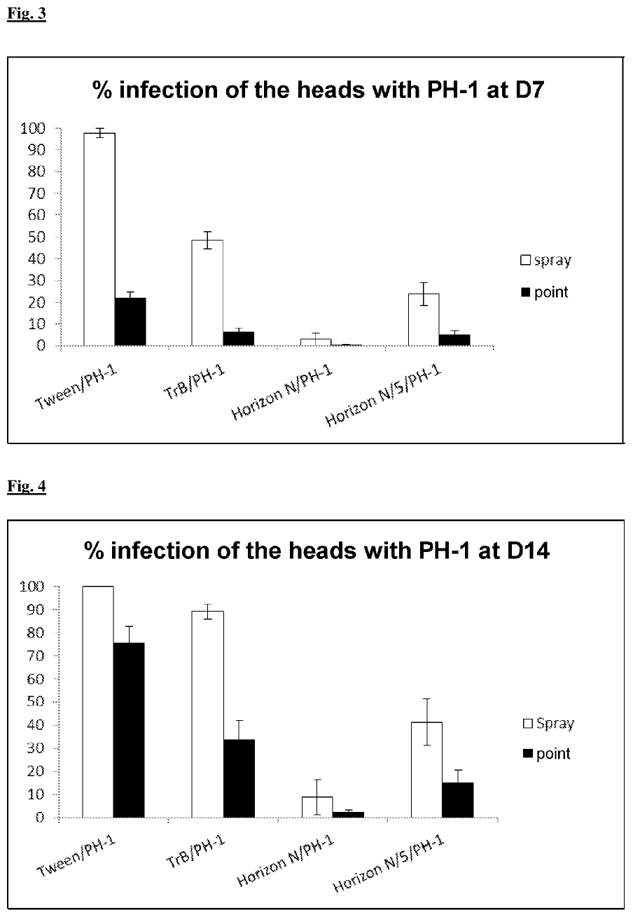

FIGS. 3 and 4 show the percentage of symptomatic spikelets on the Apogee variety of soft wheat respectively 7 days (FIG. 3) and 14 days (FIG. 4) after contamination with the strain PH-1 of Fusarium graminearum.

Tween is the uninfected control, Tr B is the Trichoderma strain of the invention and Horizon® is a commercial specialty of Bayer CropScience, the active material of which is tebuconazole 250 g/l at the approved dose (dose “N”).

The white histograms represent spray infection and the black histograms represent point infection.

FIG. 5 shows the percentage of symptomatic spikelets on the Apogee variety of soft wheat respectively at 7 days (black histograms) and at 14 days (white histograms) after contamination with the strains FG1 and INRA349 of Fusarium graminearum.

Tween is the uninfected control, Tr B is the strain of the invention. This time the Horizon® fungicide is used at one fifth of the approved dose (dose “N/5”).

The significant differences are indicated by different letters (statistical test of Mann and Whitney, α=5%). The lower-case letters (black histograms) represent the results 7 days after inoculation and the upper-case letters (white histograms) 14 days after inoculation.

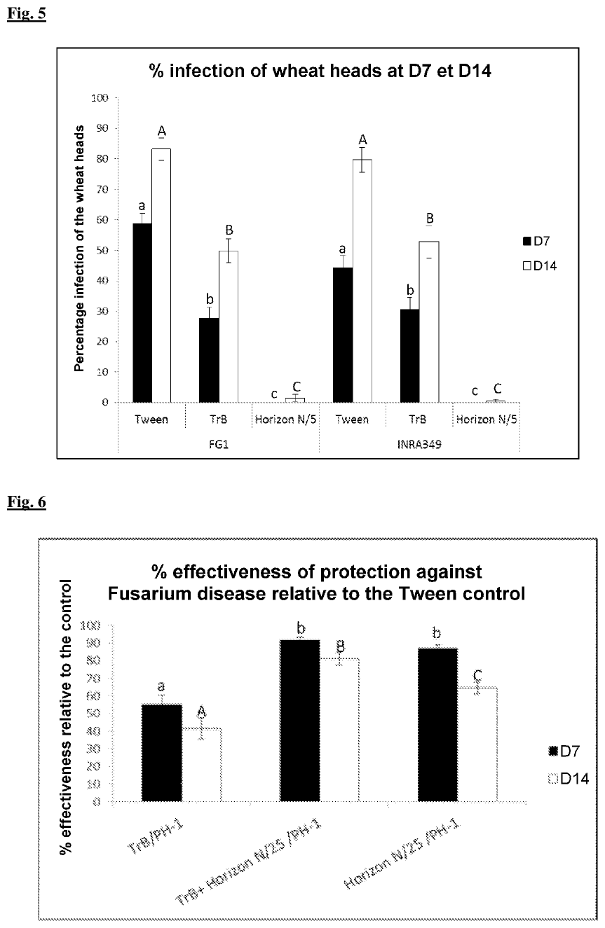

FIG. 6 shows the percentage protection conferred by the strain Tr B of the invention against Fusarium head blight on the Apogee variety of soft wheat respectively at 7 days (black histograms) and at 14 days (white histograms) after contamination with the strain PH-1 of Fusarium graminearum. For all the conditions shown, the percentage protection was calculated relative to a control (Tween).

The strain Tr B of the invention is used alone or in combination with the Horizon® fungicide, said fungicide being used this time at one twenty-fifth of the approved dose (dose “N/25”).

The significant differences are indicated by different letters (statistical test of Mann and Whitney, α=5%). The lower-case letters (black histograms) represent the results 7 days after inoculation and the upper-case letters (white histograms) 14 days after inculation.

The strains isolated and studied in the examples given hereunder are the strain of the invention Tr B and the two comparative strains Tr A (Trichoderma harzianum) and Tr C (Trichoderma longibrachiatum) as defined above.

The strain of Fusarium graminearum used in examples 2, 3 and 5 is the strain PH-1.

This strain PH-1 was selected for its aggressiveness on soft wheat, its production of mycotoxins and its chemotype of the DON/15-acetyldeoxynivalenol type (15-ADON, only 17% of acetylated form on wheat, Goswami and Kistler, 2005)⁷.

The other strains of Fusarium graminearum, namely FG1 (strain not deposited, obtained from the collection of the inventors' team) and INRA349, are used in example 4.

The strain FG1 has characteristics similar to those of the strain PH-1 (in terms of aggressiveness and production of mycotoxins of the DON/15-ADON type).

The strain INRA349 displays less aggressiveness as well as less production of mycotoxins while keeping the same chemotype of the DON/15-ADON type.

### Example 1

### Isolation and Characterization of the Trichoderma Strains Tr A, Tr B and Tr C

Various soils cultivated with soft wheats or hard wheats were sampled during summer 2015 in the Hérault Department. Core samples of small diameter (5 cm) are taken from one and the same plot, and with a depth varying from 1 to 30 cm.

Several strains of the genus Trichoderma were isolated and purified on selective medium in the laboratory with the aim of generating a collection of different strains obtained from wheat and originating from French territory.

### Sampling and Isolating Trichoderma

A small amount of earth containing organic residues is dusted and immediately dispersed in a “generalist” selective isolating medium such as Potato Dextrose Agar (PDA) kept supercooled in Petri dishes (37 to 40° C.) by stirring until solidified.

In the case of the soil organic matter obtained after suspending in water and then sieving, the suspension-dilution technique employed is as follows: different successive dilutions of the suspension obtained are incorporated in the isolating medium. For this purpose, 10 ml of each of the dilutions is poured into a conical flask containing 90 ml of selective isolating medium kept supercooled on a water bath (between 37° C. and 40° C.). After homogenizing, the 100 ml is distributed in Petri dishes placed in optimal conditions for isolating Trichoderma. After an incubation time of 3 to 7 days, the dilution having a sufficient number of colonies but without confluence is noted. Once the dilution has been selected, isolation of the colonies can be carried out.

The selective nutrient media for Trichoderma are described hereunder.

TME medium of Papavizas (1982): a mixture of glucose (1 g), agar (20 g) and distilled water (800 ml) is autoclaved. 200 ml of vegetable juice (V8® Vegetable and Fruit Juices—Campbell Soup) is added. The pH must be between 3.8 and 4. Neomycin sulfate (100 mg), bacitracin (100 mg), penicillin G (100 mg), chloroneb (100 mg), nystatin (20 mg), chlortetracycline HCl (25 mg) and sodium propionate (500 mg) are added to the medium while still lukewarm.

TSM medium of Elad et al. (1981): this medium consists of MgSO₄, 7H₂O (0.2 g); K₂HPO₄ (0.9 g); KCl (150 mg); NH₄NO₃ (1 g); glucose (3 g); chloramphenicol (250 mg); fenaminosulf (300 mg); quintozene (200 mg); rose bengal (150 mg); agar (20 g); distilled water (1000 ml).

Davet's medium (1979): Ca(NO₃)₂(1 g), CaCl₂, 2H₂O (1 g); KNO₃ (250 mg); MgSO₄, 7H₂O (250 mg); KH₂PO₄ (125 mg); sucrose (2 g); citric acid (50 mg); agar (25 g); distilled water (1000 ml). After autoclaving, the pH is adjusted to 4.5 with 1N HCl and streptomycin sulfate (30 mg), vinclozolin (2.5 mg) and allyl alcohol (0.5 ml) are added to the lukewarm medium.

The populations of Trichoderma are isolated after growing the colonies in Petri dishes at 24° C., in the light for 3 to 7 days. Purification is carried out by monospore isolation from each of the colonies (production of clones).

For each colony, the spores are taken, and then suspended in sterile water in order to dissociate them. Separate culture is carried out for each dissociated spore (under the microscope) on a rich agar medium such as PDA or oat flakes in Petri dishes.

Next, the morphological appearance of these strains during growth on various culture media is to be identified in order to determine an initial classification of the strains isolated.

The characteristics observed are the variation of the color of the colonies as a function of time, the morphological appearance of the thallus (aggregates, reverse, etc.) and conidia (shape, size, granulometry or smooth, grouped or not, etc.). Observation with the microscope at low magnification (x40) reveals the type of arborescences and their resistant forms (chlamydospores or others).

The organization of the conidiogenous structures (branchings, conidiophore, phialides, conidiospores) is studied at higher magnification.

The temperature of optimal growth of the Trichoderma clones is determined on conventional media such as malt-agar, PDA, beet pulp, oat flakes, with a pH between 4 and 7.

### Molecular Characterization of the Trichoderma Strains

Each strain was cultured in Potato Dextrose liquid medium (PD, see composition below). 20 mL of PD medium was inoculated in a 100-mL conical flask with 1 mycelial implant with a diameter of 5 mm obtained from a culture of the strain in a Petri dish of Potato Dextrose Agar medium (PDA, see composition below) aged 7 days (growth at 26° C.). Each culture was incubated for 48 h at 20° C. with stirring (120 rpm). At the end of culture, the mycelium is collected by filtration on sterile Miracloth (Calbiochem, Toulouse), dried in sterile filter paper and then frozen with liquid nitrogen and stored at −80° C. until use.

Each frozen mycelium is ground into a fine powder with liquid nitrogen and then the genomic DNA is extracted according to the protocol described by Atoui et al. (2012)⁴. The genomic DNA obtained is assayed by spectrophotometry (NanoDrop, Thermo Scientific) at 260 nm.

PCR amplification reactions are then carried out starting from 10 ng of genomic DNA from each strain of Trichoderma using two specific primer pairs: the first is specific to the EF1 gene, a unique gene coding for a translation elongation factor, and the second from a region of the ribosomal DNA, ITS1 (Internal Transcribed Spacer). The protocol used is that described by Al-Sadi et al. (2015)⁵.

The nucleotide sequence of the primers is shown in the following table:

| Name of | Nucleotide | |
|---|---|---|
| primer | sequence | Reference |
| ITS1-F | 5′-TACAACTCCCAAA | Our study |
| | CCCAATGTGA-3′ | |
| ITS1-F | 5′-CCGTTGTTGAAAG | Our study |
| | TTTTGATTCATTT-3′ | |
| EF1-728F | 5′-CATCGAGAAGTTC | (Carbone and |
| | GAGAAGG-3′ | Kohn, 1999)⁶ |
| EF1-986R | 5′-TACTTGAAGGAAC | (Carbone and |
| | CCTTACC-3′ | Kohn, 1999)⁶ |

The amplification products are then deposited on agarose gel (Tris Borate EDTA 0.5×-agarose 1.5%), purified using the NucleoSpin® Gel kit and PCR Clean-up kit (Macherey-Nagel) and ligated in the pGEM-T Easy plasmid (Promega, France), in accordance with the suppliers' recommendations. The ligation products are introduced by the thermal shock method into thermocompetent bacterial cells of the DH5a strain of Escherichia coli selected on Petri dishes of “Luria Bertani Agar” medium (“LBA”, ThermoFischer Scientifics, France) to which ampicillin is added to a final concentration of 100 μg/mL.

5 independent ampicillin-resistant clones are selected by an amplification reaction. After growth with stirring (250 rpm) at 37° C. overnight in 5 mL of Luria Bertani medium (LB, ThermoFischer Scientifics, France)+ampicillin (100 μg/mL), the cultures are deposited and then the plasmid DNA of each culture is extracted using the NucleoSpin® Plasmid kit (Macherey-Nagel).

The sequence of the PCR product is then determined by sending the plasmids for sequencing (Eurofins Genomics, Germany).

The identity of the nucleotide sequences of the 5 clones derived from the same amplification product is verified; it guarantees the purity of the strains (quality control of the purification step: in the case of amplification products of different sequences, the process is resumed starting from the purification step). In order to determine the species of each Trichoderma strain, the sequences of the amplification products specific to the EF1 gene and the ITS1 region are used as baits for searching for identical or very similar sequences in the NCBI database (https://blast.ncbi.nlm.nih.gov/) and TrichoKEY database (http://www.isth.info/tools/molkey/), respectively.

PD medium: 1 liter of tap water containing 200 g of potatoes cut into pieces is brought to the boil. The whole is filtered on carded cotton and supplemented with 20 g of glucose. The volume is adjusted to 1 liter before autoclaving for 20 minutes at 120° C.

PDA medium: same composition as the PD medium with addition of 20 g/L of agar.

### Example 2

### Efficacy of Trichoderma on the Fusarium/Brachypodium Distachyon Pathosystem in Controlled Conditions

Brachypodium distachyon (B. distachyon), studied in this example, is a monocotyledonous species in the Poaceae family, subfamily Pooideae. Its genome, relatively small for a grass, its small size, its short cycle and its taxonomic proximity to major cereals determined its choice as a model organism in genomics of the grasses.

### 1. Methodology

### Biological Material (Plants and Fungi)

Seeds of B. distachyon of the wild ecotype Bd21-3 are sterilized for ten minutes in 0.6% sodium hypochlorite while stirring gently, and then rinsed three times with sterile milli-Q® water in the same conditions.

The seeds are then kept at 4° C. for 4 days, in the dark and in sterile milli-Q® water. Sowing takes place on a 2/1 (v/v) mixture of compost and perlite, in 13-cm diameter pots at a density of 5 seeds per pot.

Growth takes place in the following conditions: 20 h of light at 24° C. and 4 h of darkness at 20° C. in order to avoid vernalization and to synchronize flowering.

The strains (Trichoderma and PH-1) are cultured on Potato Dextrose Agar medium (PDA) with addition of ampicillin and kanamycin to a final concentration of 100 and 50 μg/1, respectively, in an oven at 26° C. and are subcultured every 2 to 3 weeks.

Starting from a culture of F. graminearum (strain PH-1) aged 15 days on PDA medium, 30 ml of Mung Bean medium in a 150-ml conical flask is inoculated with 10 mycelial implants of about 5 mm² each. The medium is incubated for 7 days at room temperature, with stirring (130 rpm). At the end of this incubation, a new volume of Mung Bean is inoculated with 1/10 the volume of the first suspension of conidiospores and is incubated again for 7 days at room temperature with stirring. The suspension of spores is filtered on Sterile Miracloth and the conidiospores are counted on a Thoma cell and then resuspended in 0.01% Tween 20 (surfactant) to a final concentration of 10⁵ conidiospores/mL.

Suspensions of spores of each of the three strains of Trichoderma are prepared separately starting from a 7-day culture on PDA medium. The spores are recovered using a round-ended scalpel and then deposited in 1 ml of 0.01% Tween 20. Successive dilutions are undertaken to allow counting of the concentration of spores in the Thoma cell. Each suspension of spores Tr A, Tr B and Tr C is then adjusted to 10⁷ sp/ml in 0.01% Tween 20.

### Inoculation Technique

The efficacy in planta of the three strains of Trichoderma sp. against Fusarium head blight was visualized by spraying, until runoff, on all of the heads of B. distachyon, a suspension of spores at 10⁷ sp/ml 48 h before point inoculation of F. graminearum which, for its part, was carried out at the BBCH 65 stage on the model plant B. distachyon. 48 h after spraying of Trichoderma, point inoculation of the strain PH-1 of Fusarium graminearum is effected on the 2nd spikelet counting from the top of the head in question. 3 μl of the suspension of spores of PH-1 at a concentration of 10⁵ spores/ml is inserted between the lower and upper glumes of a flower located in the middle of the spikelet, in order to synchronize the symptoms.

The inoculated plants are then kept in the following controlled conditions: temperature 20° C., relative humidity of 65% and a cycle of short days (8 h of daylight and 16 h of darkness).

The symptoms are scored at 7, 10 and 14 days after inoculation of PH-1 based on a symptom scoring scale (see FIG. 1).

The photographs in FIG. 1 illustrate the “symptoms/score” correspondence of heads of B. distachyon (wild ecotype Bd21-3) inoculated with the strain PH-1 of F. graminearum.

### 2. Results

The results for the scores obtained 14 days after contamination with the strain PH-1 are presented in Table 1 below and in FIG. 2.

TABLE 1
Evaluation in planta of the efficacy of the three
Trichoderma sp. strains (Tr A, Tr B and Tr C) against
the strain PH-1 of F. graminearum.

| Strains | Control* | TrA | TrB | TrC |
|---|---|---|---|---|
| Mean value of the | 2.89 +/− 0.68 | 2.16 +/− 0.75 | 1.85 +/− 0.65 | 2.47 +/− 0.57 |
| scores^{α} | | | | |
| Standard error^{β} | 0.06 | 0.07 | 0.06 | 0.05 |

| | | | |
|---|---|---|---|
| p value^{δ} | <0.01 | <0.01 | <0.01 |
| % efficacy^{ε} | 25.2 | 36.2 | 14.8 |

*No preventive application of Trichoderma sp.
^{α}Mean value of the scores of the 3 independent assays +/− standard deviation
^{β}standard error = standard deviation/√(effective)
^{δ}value according to a Student test (effective > 30 in each modality)
^{ε}% efficacy = ((average score Control − average score Strain)/average score Control) × 100

### 3. Discussion

Although the various tests carried out show a significant decrease of Fusarium head blight after a preventive application of the three Trichoderma strains Tr A, Tr B and Tr C (see Table 1, FIG. 2), it is found that the best results are those obtained with the strain Tr B according to the invention.

The indigenous strain Tr B of the invention shows an efficacy above 36% (versus 25% for Tr A and 15% for Tr C).

The strain Tr B of the invention is particularly advantageous owing to its origin (since it is derived from French wheat soil) but also owing to its screening on the model cereal, Brachypodium distachyon.

In fact, screening of Trichoderma strains of interest in planta is a major advance as the majority of works described in the literature relate to selection in vitro and not in planta. The mechanisms of action of Trichoderma are complex (secondary metabolites, enzymes, stimulation of plants' defenses, competitiveness) and are in favor of selection in planta of the strains, which is difficult to carry out on wheat (too long and complex).

Advantageously, the inventors succeeded in validating the use of the model cereal B. distachyon as a means for selecting the best candidates for testing on wheat.

These results confirm the advantage of the strain Tr B of the invention for combating Fusarium head blight in cereals, and in particular Fusarium wheat head blight.

The other tests carried out hereunder are only performed on the strain Tr B according to the invention, whose efficacy is far greater than that of the comparative strains Tr A and Tr C.

### Example 3

### Tests of Efficacy in Planta of the Trichoderma Strain Tr B Against Development of Fusarium Head Blight on Spring Wheat, Variety “Apogee”

### Methodology

The seeds of spring wheat, variety Apogee, are sterilized and seeded in the same conditions as in example 2.

Moreover, the strains (Trichoderma and PH-1) are cultured in the same conditions as in example 2.

### Test in Planta

The test in planta is based on preventive inoculation of the heads with Trichoderma Tr B, 48 h before the BBCH 65 stage. Trichoderma is inoculated by spraying until runoff on wheat heads using a suspension of spores calibrated at 10⁷ spores/mL.

Two days (48 h) after supplying Trichoderma Tr B, infection with PH-1 is carried out.

Two infection techniques were tested:
(1) point infection of PH-1 is carried out between the lower and upper glumes of a flower of the 2nd spikelet, in order to synchronize the symptoms;
(2) spray infection (spraying) makes it possible to spray a titrated spore suspension on the whole head.

The plants are incubated in a climatic chamber in the following conditions: 16 h of light at 24° C.+/−5° C. under fluorescent lamp (265 μE×m⁻²×s⁻¹) at the level of the soil.

The readings of symptoms are taken as a function of time using a scoring scale by counting the symptomatic spikelets out of the total number of spikelets of each head (i.e. the percentage of symptomatic spikelets).

The results are presented in FIGS. 3 and 4.

### Discussion and Conclusion

The various tests carried out show protection of the head against Fusarium head blight by the Trichoderma strain Tr B of the invention, whether by point supply of Fusarium in a flower of each head or by spraying Fusarium on all of the heads.

At 7 days (FIG. 3), the percentage infection of the heads with PH-1 is less than 50% (against 100% for the Tween control) with TrB alone.

The strain TrB is capable of supplying significant protection against Fusarium head blight in the experimental conditions described, regardless of the manner of Fusarium contamination.

The dose of Horizon® fungicide used is the approved dose (dose “N”) (which is 250 g/l of tebuconazole). The dose N used in the controlled conditions supplies satisfactory protection at 7 days (FIG. 3) and 14 days (FIG. 4) against Fusarium head blight.

However, the dose N/5 is much less effective, since we observe:
more than 20% of infection at 7 days for N/5 (against less than 5% for N) (FIG. 3),
about 40% of infection at 14 days for N/5 (against less than 10% of infection for N) (FIG. 4).

Thus, if we envisage, for economic and ecological constraints, reducing the use of synthetic fungicides, we will easily understand the advantage of developing alternative and/or complementary solutions such as those proposed in the invention in order to obtain satisfactory protection of crops, and in particular against Fusarium head blight in wheat.

### Example 4

### Tests of Efficacy in Planta of the Trichoderma Strain Tr B of the Invention Against the Development of Fusarium Head Blight on Spring Wheat, Variety “Apogee”

### Methodology

The seeds of Apogee spring wheat variety are sterilized and seeded in the same conditions as in example 2.

Moreover, the strains (Trichoderma, FG1 and INRA349) are cultured in the same conditions as in example 2.

### Test in Planta

The test in planta is based on preventive inoculation of the heads with Trichoderma Tr B, 48 h before the BBCH 65 stage. Trichoderma is inoculated by spraying until runoff on wheat heads using a suspension of spores calibrated at 10⁷ spores/mL.

Two days (48 h) after supplying Trichoderma Tr B, infection with FG1 or INRA349 is carried out.

Infection is effected by spray (spraying) and makes it possible to spray a titrated spore suspension on the whole head.

The plants are incubated in a climatic chamber in the same conditions as in example 3.

The readings of symptoms are performed in the same conditions as in example 3.

The results are presented in FIG. 5.

### Discussion and Conclusion

We observe the same effect of protection of the Trichoderma strain TrB of the invention against the strain of F. graminearum FG1 as that usually measured against the F. graminearum strain PH-1 (52% at 7d and 40% at 14d (FIG. 5)).

However, a lowering of protection of the Trichoderma strain TrB against the F. graminearum strain INRA 349 is observed, but this protection remains constant (30-35% (FIG. 5)). This may be due to the lower virulence of this strain (narrower range of symptoms) and/or to its lower potential for production of mycotoxins. In fact, on the assumption that the TrB strain could exert an action on the production of mycotoxins of the Fusarium strains, this effect will be far more visible on a strongly producing strain that on a weakly producing strain.

Thus, if we envisage, for economic and ecological constraints, reducing the use of synthetic fungicides, we will easily understand the advantage of developing alternative and/or complementary solutions such as those proposed in the invention in order to obtain satisfactory protection of crops, and in particular against Fusarium head blight in wheat, extensively with different strains of the causal agent.

### Example 5

Tests of efficacy in planta of a combination of the Trichoderma strain Tr B and reduced dose of Horizon® fungicide against development of Fusarium head blight on spring wheat, variety “Apogee”

### Methodology

The seeds of spring wheat variety Apogee are sterilized and seeded in the same conditions as in example 2.

Moreover, the strains (Trichoderma, PH-1) are cultured in the same conditions as in example 2.

The mixture of spores of Trichoderma Tr B (suspension of spores calibrated at 10⁷ spores/mL) and of the fungicide of the azole type at the dose N/25 is prepared extemporaneously in water.

### Test in Planta

The approved dose of Horizon® fungicide used is the dose “N” (which is 250 g/l of tebuconazole). The dose “N” used in the controlled conditions supplies complete protection, not allowing differences to be visualized. The dose used for quantifying the respective supply of Trichoderma Tr B and Horizon® fungicide was determined at “N/25”.

The test in planta is thus based on preventive inoculation of the heads, 48 h before the BBCH 65 stage, with:
the strain of the invention Tr B alone,
the mixture Trichoderma Tr B/Horizon® (dose “N/25”),
the Horizon® fungicide alone (dose “N/25”).

The wheat heads are inoculated with these different preparations by spraying until runoff.

Two days (48 h) after supplying each preparation separately, infection with PH-1 is carried out.

Infection is carried out by spray (spraying) and makes it possible to spray a titrated spore suspension on the whole head.

The plants are incubated in a climatic chamber in the same conditions as in example 3.

The readings of symptoms are performed in the same conditions as in example 3.

The results are presented in FIG. 6.

### DISCUSSION AND CONCLUSION

It is observed that the Trichoderma strain Tr B of the invention alone confers protection against Fusarium head blight of 55% at 7 days after inoculation and of 40% at 14 days after inoculation (FIG. 6).

The Horizon® fungicide at the “N/25” dose confers protection of 88% at 7 days after inoculation and of 65% at 14 days (FIG. 6).

It is observed that the mixture of the Trichoderma strain Tr B of the invention with the “N/25” dose of Horizon® fungicide supplies an efficacy of protection of 90% at 7 days after inoculation and of 80% at 14 days after inoculation (FIG. 6).

This efficacy of protection of the mixture of the Trichoderma strain Tr B of the invention with the “N/25” dose of Horizon® fungicide is enhanced at 14 days after inoculation compared to the Horizon® fungicide alone at the “N/25” dose.

Thus, if we envisage, for economic and ecological constraints, reducing the use of synthetic fungicides, we will easily understand the advantage of developing complementary solutions such as those proposed in the invention in order to obtain satisfactory protection of crops, and in particular against Fusarium head blight in wheat.

1-13. (canceled)

14. A Trichoderma harzianum strain deposited on Jun. 12, 2018 with the National Collection of Cultures of Microorganisms (CNCM), 25-28 rue du Docteur Roux, 75724 Paris Cedex 15, under number CNCM I-5327.

15. A method of combating Fusarium head blight of cereals, comprising applying the Trichoderma harzianum strain as claimed in claim 14 onto a cereal selected from the group comprising wheat, Brachypodium distachyon, triticale, oat, rye and maize, and preferably wheat.

16. The method as claimed in claim 15, in which Fusarium head blight is caused by a fungal phytopathogen of the genus Fusarium and/or Microdochium.

17. The method as claimed in claim 15, wherein the Fusarium head blight is Fusarium wheat head blight.

18. The method as claimed in claim 15, wherein the Trichoderma harzianum strain decreases a content of mycotoxins present in wheat, said mycotoxins in particular being deoxynivalenol (DON).

19. The method as claimed in claim 15, wherein the Trichoderma harzianum strain is applied at the level of the cereal head, and preferably the wheat head.

20. The method as claimed in claim 15, wherein the Trichoderma harzianum strain is applied starting from the beginning of flowering of the cereals and until full flowering of the cereals.

21. The method as claimed in claim 15, wherein the Trichoderma harzianum strain is applied on the cereals, at a concentration of spores ranging from 10⁶ to 10⁹ spores/ml, and preferably from 10⁷ to 10⁸ spores/ml, in a volume of 100 to 200 liters per hectare.

22. The method as claimed in claim 15, wherein the Trichoderma harzianum strain is combined with a fungicide of the azole type selected from the group comprising prothioconazole, tebuconazole, metconazole, mefentrifuconazole and mixtures thereof.

23. The method as claimed in claim 22, wherein the Trichoderma harzianum strain separate or sequential administration with said fungicide.

24. The method as claimed in claim 22, wherein the amount of fungicide is from 2% to 60%, and preferably from 4% to 35% of the recommended dose for effective action for combating Fusarium head blight.

25. A composition for combating Fusarium head blight in cereals, comprising:
a Trichoderma harzianum strain as defined in claim 14, and
a formulating agent and/or an adjuvant.

26. A kit, comprising, separately:
a Trichoderma harzianum strain as defined in claim 14,
a fungicide of the azole type selected from the group comprising prothioconazole, tebuconazole, metconazole, mefentrifuconazole and mixtures thereof.


SEQUENCE LISTING (4 sequences)

SEQ ID NO: 1 - DNA, 23 bp, artificial sequence
  synthetic oligonucleotide primer ITS1-F
tacaactcccaaacccaatgtga

SEQ ID NO: 2 - DNA, 26 bp, artificial sequence
  synthetic oligonucleotide primer ITS1-F
ccgttgttgaaagttttgattcattt

SEQ ID NO: 3 - DNA, 20 bp, artificial sequence
  synthetic oligonucleotide primer EF1-728F
catcgagaagttcgagaagg

SEQ ID NO: 4 - DNA, 20 bp, artificial sequence
  synthetic oligonucleotide primer EF1-986R
tacttgaaggaacccttacc